(19) United States
(12) Patent Application Publication
(10) Pub. No.: US 2009/0125023 A1
(43) Pub. Date: May 14, 2009

(54) Electrosurgical Instrument

(75) Inventors: Arthur Stephen, Raynham, MA (US); Daniel Beaudet, Boston, MA (US); David Freed, Westborough, MA (US); Michael Graffeo, Millbury, MA (US); Luis Pedraza, West Roxbury, MA (US); Jay O'Keefe, North Attleboro, MA (US)

Correspondence Address: CYTYC CORPORATION;Darry Pattinson, Sr. IP Paralegal, 250 CAMPUS DRIVE, MARLBOROUGH, MA, 01752, US

(73) Assignee: Cytyc Corporation

(21) Appl. No.: 11/938,989

(22) Filed: Nov. 13, 2007

(51) Int. Cl. A61B 18/14

(52) U.S. Cl. 606/42

(57) ABSTRACT

Surgical instruments include a handle, gas and electrical conduits, a hollow elongated electrode, elongated insulating sheath, and actuator mechanism. The handle has distal and proximal ends, the proximal end branching to form two arm-like portions and the distal end widening to form a tubular body portion having features sized for receiving at least an index finger and a thumb. Gas and electrical conduits are disposed within the handle for connection to external gas and electrical sources. A hollow elongated electrode extends from the handle, defines a gas conduit along its length, and is operably coupled to the gas and electrical conduits. An elongated insulating sheath encloses the electrode. An actuator mechanism alternates between two states to turn the instrument on and off. A sheath actuator slidably retracts and extends the elongated insulating sheath to increase or decrease exposure of the electrode. Methods for operating electrosurgical instrument are also disclosed.

### TECHNICAL FIELD

Surgical instruments and methods of operating same to perform least invasive surgical techniques in the medical field.

### BACKGROUND

Surgical and interventional instruments for use in least invasive surgical procedures are disclosed herein. Least invasive surgical (LIS) techniques, such as laparoscopy, endoscopy, artheroscopy, thoracoscopy, and pelviscopy, are generally performed through small incisions using specialized instruments to perform desired surgical procedures. Typically, the surgical instruments are introduced through a tube, such as a cannula or trocar sleeve, while the physician observes manipulation of the instruments through specialized imaging equipment, such as laparoscopes, endoscopes, thoracoscopes, and artheroscopes. During LIS procedures it is frequently necessary to cauterize, coagulate, ablate, sever, or otherwise manipulate tissue using an electrosurgical instrument.

Electrosurgical instruments apply electrical energy to body tissue to change the structure or function of the tissue or body organ. Electrosurgical instruments apply high frequency current to excise tissue and/or close small bleeding blood vessels by coagulation. Electrosurgical procedures are particularly advantageous since they reduce bleeding from small blood vessels, facilitating the handling of highly vascularized tissues while minimizing exposure of the patient to shock and pain.

Electrosurgical instruments are operated by a surgeon and during some surgical procedures, may be held in a surgeon's hand for several hours at a time. Typical electrosurgical instruments have a straight pencil-like shape and may be held like a pencil (resting middle finger and held by thumb and index finger) or may be completed engulfed in the hand in a dagger-like grip (thumb on top and all other fingers underneath). It would be desirable to have an instrument handle providing a more comfortable and natural grip position, such as an ergonomic handle. Disclosed herein are ergonomic handles which provide a more comfortable and natural hand position for the surgeon during operation. These ergonomic handle may also provide a surgeon with more precise fine-tuned control over the instrument during operation, as well as a variety of different hand positioning options.

Electrosurgery may be performed by using an electrosurgical instrument coupled togas and electrical power sources to generate an ionized stream, also known as an ionized gas plasma flame or stream, for use in coagulation. Electrosurgical instruments for generating an ionized gas plasma stream typically include a handle grip, an electrode, external power and gas sources, and a switch or foot-pedal coupled to the external power source for turning the device on/off.

An important consideration in the design of currently available electrosurgical instruments has been the placement of the on/off switch and other switches, which must be manually activated by the surgeon in order to turn the instrument on/off or change operational modes during use. The placement of these switches is important for providing both comfortable and easy to access during operation, while also being positioned so as to prevent the accidental activation of the switch during operation. It would therefore be desirable to have an electrosurgical instrument having an ergonomic handle and conveniently and comfortably located switches.

### SUMMARY

Disclosed herein are electrosurgical instruments and methods for operating electrosurgical instruments. The electrosurgical instruments disclosed herein have ergonomical handles to provide easier and more controlled manipulation of the device. These electrosurgical instruments may be used in the medical surgical field and may be advantageous for use in laparoscopic surgical procedures. The ergonomic handles of the electrosurgical instruments help give a surgeon better fine tuned control of the instrument, helping the surgeon achieve more precise positioning and manipulation of the instrument during operation. Additionally, the instrument handle may provide a more comfortable and natural position for surgeon's hand. Because some surgical procedures may be quite lengthy and take hours to complete, a more comfortable and natural positioning of the surgeon's hand may help to prevent discomfort and cramping.

These electrosurgical instruments may comprise an actuator mechanism to turn the instrument on and off, as well as a sheath actuator to change the operating mode (i.e., cutting, coagulation, etc.) of the instrument during use. The actuator mechanism and sheath actuator may be collectively referred to as actuation mechanisms herein. These actuation mechanisms may be conveniently positioned on the handle of the instrument and may further be positioned in particular areas on the handle to make it comfortable for the surgeon to access. In one embodiment, the handle may have concave surfaces sized for receiving a finger or a thumb and the actuation mechanisms may be positioned within these concave surfaces to provide convenient and comfortable access to these mechanisms during operation. The ability to easily access the actuation mechanisms during use provides more efficient operation for a surgeon, eliminating the need to change grip or hand-positioning to reach the actuation mechanism and also eliminates the need for additional foot-pedal coordination.

In one embodiment, an electrosurgical instrument includes an electrically insulated handle, gas and electrical conduits, a hollow elongated electrode, an elongated insulating sheath, and an actuator mechanism. The electrically insulated handle has distal and proximal ends. The proximal end branches to form two arm-like portions and the distal end widens to form an approximately tubular body portion having features sized for receiving at least an index finger and a thumb. The gas and electrical conduits are disposed within the handle for connection to external gas and electrical sources. The hollow elongated electrode extends from the distal end of the handle and defines a gas conduit along its length. The elongated electrode is operably coupled to the gas and electrical conduits. The elongated insulating sheath encloses the elongated electrode. The actuator mechanism is operably coupled to the handle, the elongated electrode, and to the gas and electrical conduits. The actuator mechanism is configured to alternate between at least two states; wherein a first state prevents electric current and gas from reaching the elongated electrode, and wherein a second state allows electric current and gas to reach and flow through the elongated electrode to generate an ionized plasma gas stream for electrosurgery.

In one embodiment, the electrosurgical instrument may further comprise a sheath actuator disposed within and extending from the distal tip of the handle. The sheath actuator may surround and be operably coupled to the elongated insulating sheath. The sheath actuator may be configured to slidably retract and extend the elongated insulating sheath to increase or decrease exposure of the elongated electrode. When the sheath actuator is retracted it will increase exposure of the elongated electrode, resulting in a cutting mode of operation. When the sheath actuator is extended it will decrease exposure of the elongated electrode, resulting in a coagulation mode of operation.

In yet another embodiment, a method of operating an electrosurgical instrument is disclosed. The method includes grasping a handle having distal and proximal ends, wherein the proximal end branches to form two arm-like portions and the distal end widens to form an approximately tubular body portion. The tubular body portion has surfaces sized for receiving a middle finger and a thumb and the branch between the two arm-like portions is sized for receiving an index finger. The method continues by activating an actuator mechanism positioned on the handle using at least one of the middle finger, thumb, or index finger to operate the electrosurgical instrument.

This summary is provided to introduce a selection of concepts in a simplified form that are further described below in the detailed description. This summary is not intended to identify key features or essential features of the claimed subject matter, nor is it intended to be used to limit the scope of the claimed subject matter. It should also be understood that, although electrosurgical instrument implementations are described here, the described technology may be applied to other systems.

### BRIEF DESCRIPTION OF THE DRAWINGS

FIG. 1A illustrates a perspective view of an exemplary electrosurgical instrument;

FIG. 1B illustrates a perspective view of an exemplary electrosurgical instrument held within a user's hand;

FIG. 2A illustrates a top view of an exemplary handle of an. electrosurgical instrument;

FIG. 2B illustrates a side view in elevation of an exemplary handle of an electrosurgical instrument;

FIG. 3 illustrates a partial cross-sectional view in elevation of an exemplary electrosurgical instrument;

FIG. 4 is an electrical circuit diagram of an exemplary electrosurgical instrument;

FIG. 5A illustrates a perspective view of an exemplary electrosurgical instrument having a sheath actuator and showing the sheath retracted;

FIG. 5B illustrates a perspective view of an exemplary electrosurgical instrument having a sheath actuator and showing the sheath extended;

FIG. 6 illustrates a perspective view of an exemplary electrosurgical instrument having a wheel mechanism sheath actuator;

FIG. 7A illustrates a top view of an exemplary electrosurgical instrument having the two arm-like portions coupled at proximal ends for form a loop;

FIG. 7B illustrates a side view of FIG. 7A; and

FIG. 8 is a flow chart illustrating exemplary operation of an electrosurgical instrument.

### DETAILED DESCRIPTION

Disclosed herein are surgical instruments and methods for operating surgical instruments. These instruments may be used in medical procedures, such as laparoscopic surgical procedures, electrosurgical procedures, or open surgical procedures. The electrosurgical instruments described herein may operate to generate an ionized plasma gas stream for performing coagulation, cutting, or cauterization procedures. Electrosurgical instruments for generating an ionized plasma gas stream are also described in U.S. Pat. No. 6,255,593, entitled Medical Apparatus for Generating and ionized Gas Plasma Stream, which is incorporated by reference herein for all that it discloses.

The electrosurgical instruments disclosed herein may be manually or hand-operated by a surgeon. The electrosurgical instruments have an ergonomical handle to provide easier and more controlled manipulation of the device. The ergonomic handles may help a surgeon to more precisely and accurately control and manipulate the instrument during operation. The ergonomic handle may be grasped using a number of different types of hand gripping positions, including finger tip control. Additionally, the ergonomic handle may provide a more comfortable and natural position for a surgeon's hand to help prevent hand cramping or discomfort during operation.

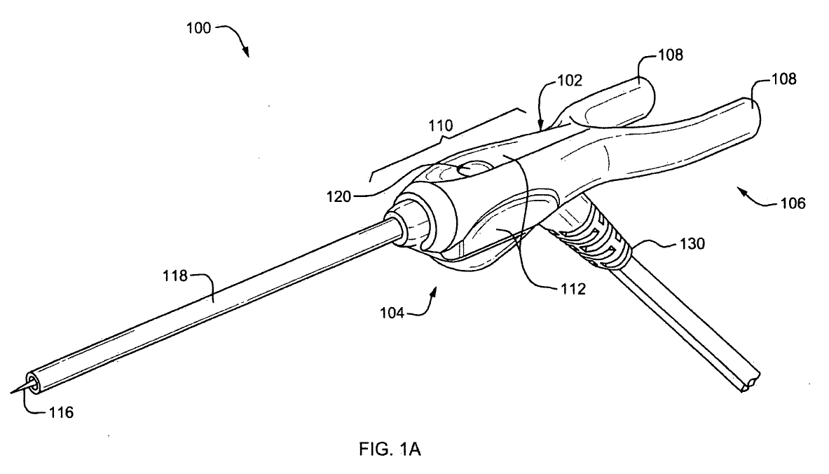

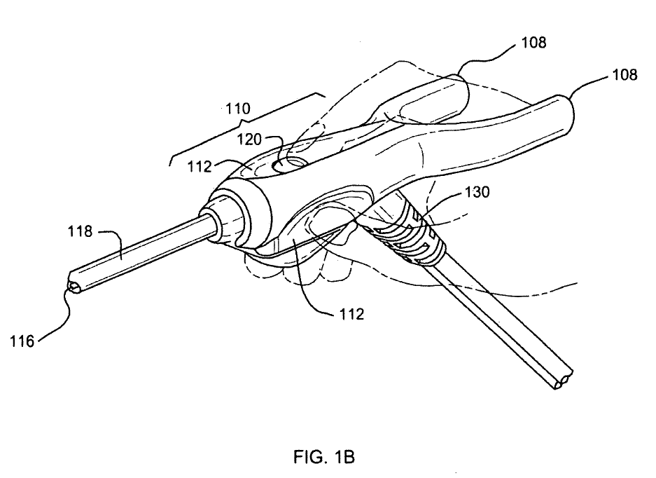

FIG. 1A illustrates a perspective view of an exemplary electrosurgical instrument 100. An exemplary electrosurgical instrument 100 includes an electrically insulated handle 102, a hollow elongated electrode 116, an elongated insulating sheath 118, gas and electrical conduits, and an actuator mechanism 120.

The electrically insulated handle 102 has distal 104 and proximal 106 ends. The proximal end 106 branches to form two arm-like portions 108 and the distal end 104 widens to form an approximately tubular body portion 110 having features 112 sized for receiving at least an index finger and a thumb, as shown in FIG. 1B. As shown in FIGS. 1A and 1B, the approximately tubular body portion 110 may have an enlarged ‘belly’ on the underside of the handle to provide a larger surface area for the fingers and thumb and to provide better comfort and control when articulating the handle 102. In some implementations, the tubular body portion 110 may have an approximately triangular shape when viewed as a cross-section because of the indentations or recessed features 112 disposed within the tubular body portion 110. When viewed as a cross-section, the thumb, index finger, and middle finger may each be positioned on one of the three sides of the triangularly shaped body portion.

The handle 102 will be formed of an electrically insulating material to protect a user, such as a plastic or elastomeric material. In some embodiments, the handle 102 may be formed from a spongy, softer, or resilient material to increase user comfort. The surface of the handle 102 may also be coated with a softer, textured, patterned, or tacky material to provide better tactile feedback and user comfort and control. In some implementations, the handle 102 may be formed of a variety of different types of materials. For example, the recessed or concave areas 112 may be formed of a softer material while the remaining areas of the handle 102 may be formed of a more rigid material. Handle 102 may be formed of a number of different materials, such as polyphenylsulfone, polycarbonate, nylon, ABS, polystyrene, polyetherimide, and polyphenyleneoxide, for example.

With continuing reference to FIGS. 1A and 1B, the features 112 within the tubular body portion 110 may comprise concave areas sized for receiving an index finger, middle finger, and/or thumb. The features 112 may be formed as a slight recess or may have a significant recess for more securely enclosing and/or partially surrounding a finger tip. In some implementations, the features 112 may simply be formed of a different material and may not necessarily be recessed or concave. In some implementations, the features 112 may be only partially recessed.

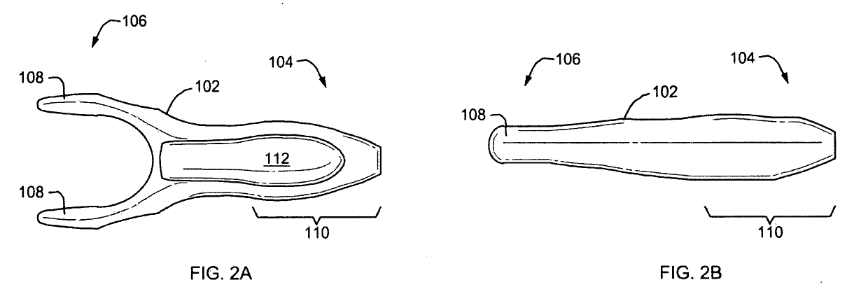

As shown in FIGS. 2A and 2B, the two arm-like portions 108 branch away from the tubular body portion 110 to form a generally Y-shaped handle 102. The two arm-like portions 108 may branch away from tubular body portion 110 to form a U-shape or a Y-shape therebetween. The arm-like portions 108 may be formed to have shorter or longer lengths, thinner or thicker portions, may be curved, slightly curved, or straight, and may be rigid or flexible. The arm-like portions 108 are thin enough to fit between the index and middle fingers with no discomfort. The arm-like portions 108 may be curved slightly inward toward a center axis of handle 102 to wrap slightly around the index finger to more securely couple the handle 102 within a user's grasp.

The outer surface of handle 102 may be formed of smooth rounded contours, as shown in FIG. 1, or may be formed of more straight contours, as shown in FIG. 2A. The space between the arm-like portions 108 is sized for receiving an index finger therein. The index finger may then rest in recessed or concave feature 112, as shown in FIG. 1B. The middle finger and thumb may rest against the recessed or concave features 112 along the sides or belly of tubular body portion 110, as shown in FIG. 1B.

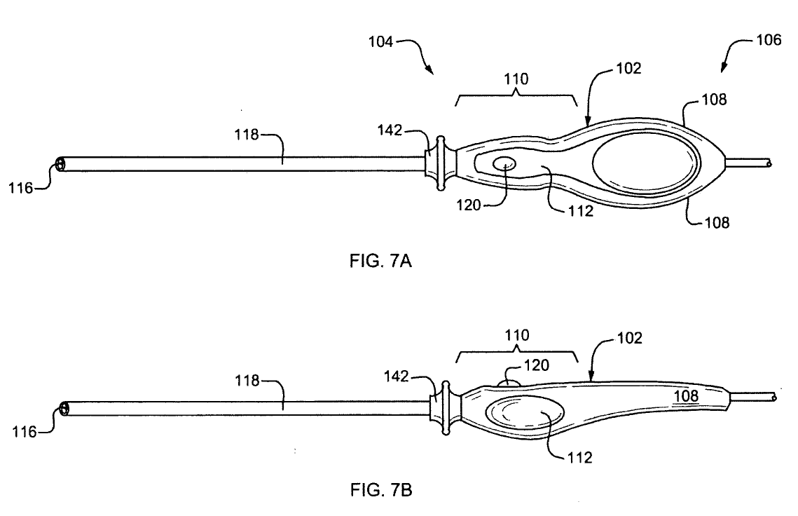

In one embodiment, the two arm-like portions 108 may be coupled or joined together at proximal end 106 to form a loop (shown as 108), as shown in FIG. 7A. In this implementation, the loop or ring formed by the two arm-like portions 108 is sized to receive an index finger therein. This implementation may be advantageous for allowing a user to stretch his/her hand during operation, as the entire hand may be fully opened and the user can still maintain control of the instrument 100 using only an index finger. FIG. 7B illustrates a side view of this embodiment.

The ergonomic electrosurgical instrument handles 102 disclosed herein are intended to maximize productivity and control by reducing operator fatigue and discomfort. The handle 102 may be grasped by a user using a classic grip, in which the handle is secured between the thumb and middle finger (with the index finger resting on top), as shown in FIG. 1B. When the handle 102 is held using this classic grip, a user can open or stretch his/her hand while still having control of the instrument 100. The shape of the handle 102 and positioning of the actuator mechanism 140 (described in more detail below) are conveniently located for use of either a classic grip or a dagger grip, so a user can select his/her preferred grip and/or can change grip during a procedure to prevent fatigue.

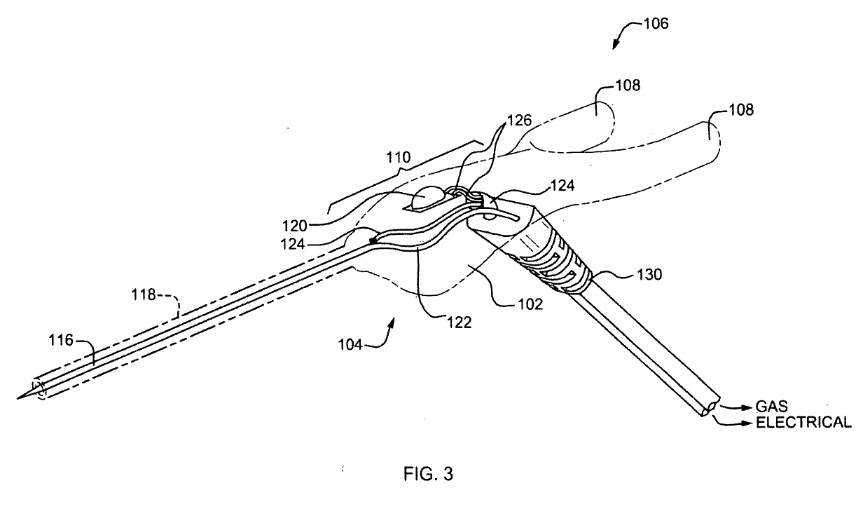

FIG. 3 illustrates a partial cross-sectional view in elevation of an exemplary electrosurgical instrument 100. Disposed within handle 102 are a gas conduit 122 and an electrical conduit 124. The gas 122 and electrical 124 conduits are operably coupled to elongated electrode 116 as well as to external gas and electrical sources (not shown). The gas may be an inert gas, such as helium. The electrical or power source may be RF energy. When both the gas and electrical sources are activated or turned “on” an ionized gas plasma stream is generated from the distal tip of the electrode 116.

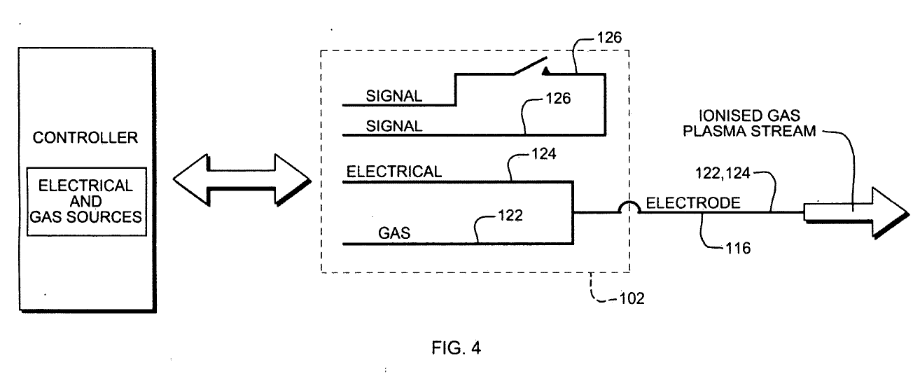

FIG. 4 is an electrical circuit diagram illustrating the gas 122 and electrical 124 pathways within handle 102 and operably coupled to electrode 116 and to external electrical and gas sources within an external controller (note that a gas source/tank may be separate from controller but operably coupled to instrument 100).

Specifically, the external electrical source may be a fixed frequency power supply which initiates and maintains a low power plasma stream discharge. This may be achieved by using a resonant circuit that is resonant at a fixed frequency so that when the plasma stream is formed, the voltage is reduced because of the impedance provided by the plasma stream relative to the resonant circuit, thereby reducing the current flow and the power delivered to the plasma stream.

Further, the electrosurgical instrument 100 may have a monopolar electrode arrangement. In a monopolar electrode arrangement, a single electrode 116 is energized and electric current is directed through the patient between the electrode 116 and a dispersive pad or plate upon which the patient is placed or which is attached to the patient (not shown).

External gas and electrical sources may be located within an external controller or may be coupled to an external controller. The controller may be responsive to the electrosurgical instrument 100 via the actuator mechanism 120 to allow easy operator adjustment of the external gas source/flow and the external electrical power source. The controller is not shown herein for simplicity, but will be understood by those of ordinary skill in the art after having become familiar with the teachings herein.

External gas and electrical sources may be operably coupled to handle 102 via coaxial or biaxial lines, which may be surrounded by strain relief 130 at the point where the cables are attached to the handle 102, as shown in FIGS. 1 and 3. The strain relief 130 prevents damage to insulation of the gas and electrical lines during manipulation of the instrument 100. In alternative embodiments, the strain relief 130 may be attached to the handle 102 at a different location, such as on one of the two arm-like portions 108, as shown in FIGS. 7A and 7B. In some implementations it may be desirable to couple the gas and electrical lines directly to the handle 102 without use of a strain relief 130.

Both the gas 122 and electrical 124 conduits are coupled to the hollow elongated electrode 116. The hollow elongated electrode 116 is formed an electrically conductive material and provides an elongated channel or gas conduit to carry gas along the length of the electrode 116. The gas conduit 122 may be directly coupled to the electrode 116 to form a continuous conduit or channel for carrying the gas to the distal tip of the electrode 116. The electrical conduit 124 may be electrically coupled to the electrode 116, such as by soldering, crimping, and/or a type of interference fit. The electrical conduit 124 couples the electrical power source to the elongated electrode 116, energizing the electrode 116 during operation.

The elongated electrode 116 is enclosed by an insulating sheath 118 to protect a user and patient from electrical exposure during operation. The tip of electrode 116 may be sharpened to a point to provide a ‘cutting’ mode of operation when the electrode 116 is exposed (and placed into direct contact with tissue), as shown in FIG. 3. In one embodiment, the tip of electrode 116 may be formed as a separate electrically conductive component and coupled to the distal tip of electrode 116. The amount of electrode 116 exposed may be varied by extending or retracting the insulating sheath 118, as will be described in more detail below.

The electrosurgical instrument 100 also includes an actuator mechanism 120 operably coupled to the handle 102, the elongated electrode 116, and to the gas 122 and electrical conduits 124. The actuator mechanism 120 is configured to alternate between at least two states. The actuator mechanism 120 is operably coupled to control circuitry in an external controller (via gas and electrical lines) or generator, which enables or inhibits gas and electrical flow through the gas 122 and electrical 124 conduits to electrode 116. In one embodiment, the actuator mechanism 120 may also be coupled to a valve in an external controller, the valve being operable between open and closed states to turn the gas on and off.

When the actuator mechanism 120 is in a first state, the passage or flow of gas and electric current through the elongated electrode 116 will be inhibited. When the actuator mechanism 120 is in a second state it will enable the passage or flow of gas and electric current through the elongated electrode. In this second state, the gas and electrical power flow may be simultaneously activated by actuator mechanism 120 via control circuitry in an external controller.

In the first state the electric current and gas flow do not reach or energize the electrode 116, resulting in the inactivation or “off” state of the instrument 100. In the second state, both the electric current and gas flow reach and flow through the electrode 116 via electrical pathway 124, to energize electrode 116 while gas is flowing through the electrode 116, resulting in the formation of an ionized plasma gas stream or “on” state of the instrument 100.

The actuator mechanism 120 may comprise a number of different types and configurations of actuators, such as a push-button, slide, wheel, or other mechanism. FIGS. 1, 3, 5A & 5B illustrate a push-button actuator mechanism 120, however may other configurations are contemplated.

As shown in FIGS. 3 and 4, the actuator mechanism 120 is coupled to two electrical signal lines 126. The electrical signal lines 126 are operably coupled to electrical conduit 124 and are configured to communicate with electrical conduit 124 to alternate between the first and second states to turn the device on or off. For safety purposes, it may be desirable to configure or bias the actuator mechanism 120 and electrical signal lines 126 so that the instrument 100 remains in the “off” position until the actuator mechanism 120 is activated.

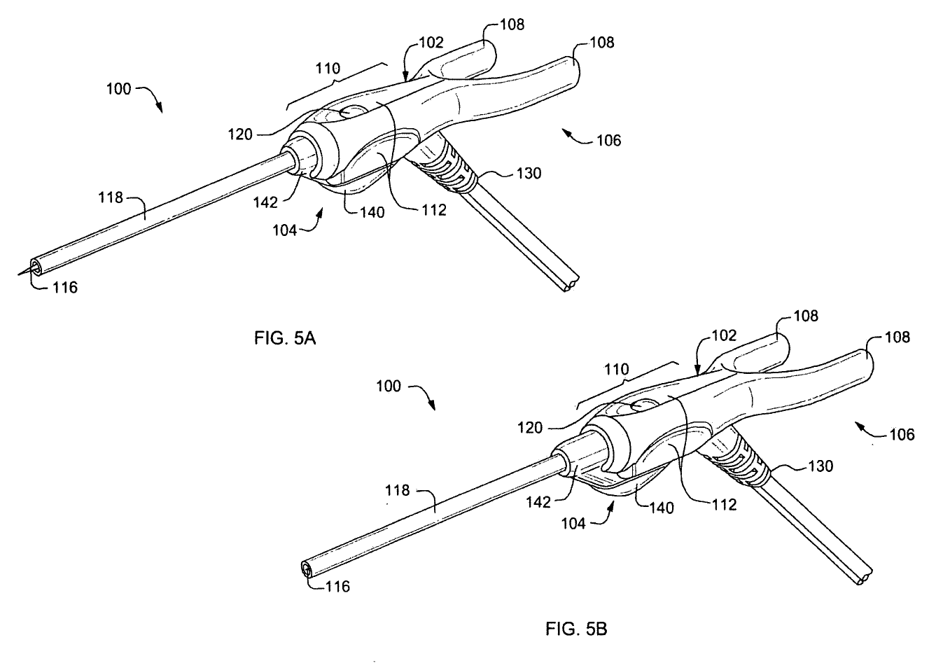

The instrument 100 may further comprise a sheath actuator 140 disposed within and extending from the distal end 104 of the handle 102. FIG. 5A illustrates a sheath actuator 140 retracted and FIG. 5B illustrates a sheath actuator 140 extended. The sheath actuator 140 may be a collar 142 which surrounds and is operably coupled to the elongated insulating sheath 118, to slidably retract and extend the elongated insulating sheath 118 to increase or decrease exposure of the elongated electrode 116.

In some implementations the sheath actuator 140 may simply be the elongated insulating sheath 118. In this implementation, the elongated insulating sheath 118 may be manually slid to increase or decrease exposure of the electrode 116. The sheath actuator 140 may simple be an enlarged section of the insulating sheath 118 which is easier for a user to grasp and slide. In an alternative implementation, the elongated insulating sheath 118 may remain stationary while the elongated electrode 116 itself may be extended or retracted.

In other implementations, the sheath actuator 140 may be a collar 142, as shown in FIGS. 5A and 5B, which is operably coupled to the elongated insulating sheath 118. The collar 142 may be sized to be slidably received within the distal end 104 of the handle 102. The collar 142 slides inside the distal end 104 of the handle 102 to eliminate potential pinch-points and prevent electrical leakage. The collar 142 may also be coupled to a switch, a slide, a rotatable mechanism, or a wheel to provide more convenient thumb or finger activation of the collar 142 or sheath actuator 140 to extend or retract insulating sheath 118.

The sheath actuator 140 may also be a switch, slide, rotatable mechanism, or a wheel. The sheath actuator 140 may be positioned at any number of different locations on handle 102 and may be operable via a thumb or finger. In some implementations, multiple sheath actuators 140 may be provided for alternating between multiple modes of operation. In other implementations, a single sheath actuator 140 may be present at more than one location or position on the handle 102, to provide easy access to the user when the handle 102 is held in different grips. For example, sheath actuator 140 may be a slidable switch having two separate switch faces, either of which may be activated by the thumb or middle finger. In yet another embodiment, the actuator mechanism 120 and sheath actuator 140 may be integrated into the same mechanism having a plurality of different positions operable to alternate between on/off as well as modes of operation.

As shown in FIG. 5B, the collar 142 may be coupled to a slide mechanism 140 which may be operated by a thumb to extend the collar 142 which extends the insulating sheath 118, which minimizes or decreases exposure of the electrode 116 to allow operation of the instrument 100 in a coagulation mode of operation. FIG. 5A illustrates the instrument 100 with the thumb slide mechanism 140 and collar 142 retracted to retract the insulating sheath 118 to increase exposure of the electrode 116 to allow operation of the instrument 100 in a cutting mode of operation which the electrode 116 is in direct contact with tissue.

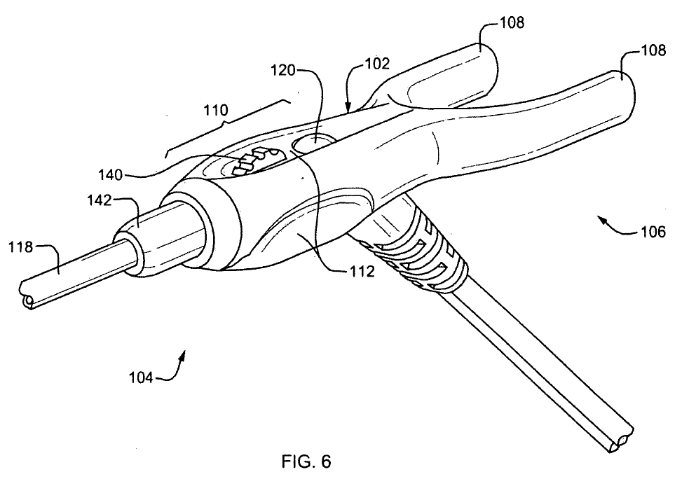

FIG. 6 illustrates an alternative embodiment, wherein the actuator mechanism 140 comprises a wheel 140. In this implementation, the wheel 140 may be operated by the index finger resting on top of the tubular body portion 110 of handle 102. The wheel 140 may have multiple positions (similar to a wheel on a computer mouse) which may be adjusted or fine-tuned by a user to provide an adjustable mechanism for controlling extending and retracting of insulating sheath 118 to control exposure of electrode 116. In this implementation, a user may choose to expose only a very minimal amount of electrode 116 to limit or more precisely control the cutting mode of operation, forming a partial cutting mode of operation.

In some embodiments, it may be desirable to have an electrosurgical instrument 100 without a sheath actuator 140, as shown in FIG. 1. In this embodiment, the elongated insulating sheath 118 may be permanently affixed to the elongated electrode 116 to decrease direct exposure of the electrode 116 to tissue, resulting in an instrument having a predetermined coagulation mode of operation. In an alternative embodiment, it may be desirable to have an electrosurgical instrument 100 capable of alternating between a coagulation mode and a cutting mode of operation. In order to alternate between a coagulation mode and a cutting mode of operation, the direct exposure of the electrode 116 to tissue may be altered.

When the electrode 116 is fully or substantially recessed within the insulating sheath 118 (by extending the insulating sheath 118 over the electrode 116) the instrument 100 is operable in a coagulation mode, as shown in FIG. 5B. When the electrode 116 extended beyond the insulating sheath 118 (by retracting the insulating sheath 118 to expose the electrode 116) the sharpened distal tip of the electrode 116 may be manipulated to directly contact and cut tissue for use in a cutting mode of operation, as shown in FIGS. 1 and 5A.

The electrosurgical instrument handle 102 disclosed herein allows a user to have better fingertip control of the actuator mechanism 120 and sheath actuator 140, as well as of the entire instrument 100. The locations of the actuator mechanism 120 and sheath actuator 140 on the handle 102 have been optimized to allow for comfortable and convenient one-handed activation. The optimal positioning of the actuator mechanism 120 and sheath actuator 140 eliminates the need for use of a foot-pedal or a second hand, however in some implementations is may still be desirable to use a foot-pedal. The actuator mechanism 120 and sheath actuator 140 may also be configured to have a predetermined level of resistance to activation if positioned directly in typical finger resting or operating position to prevent accidental or inadvertent activation.

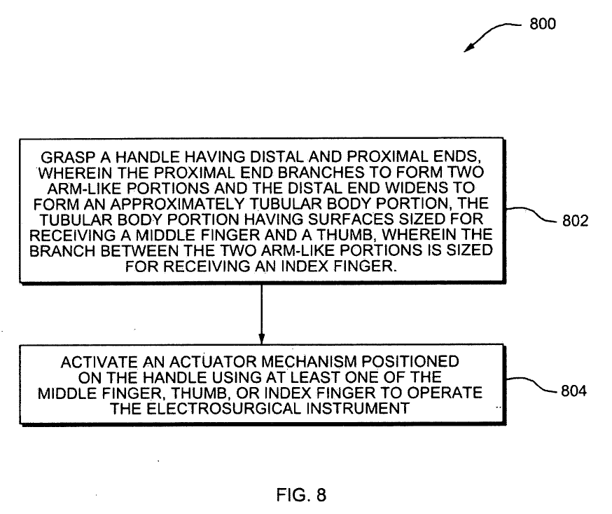

A method 800 of operating an electrosurgical instrument 100 is also disclosed herein, as shown in FIG. 8. In some implementations, the electrosurgical instrument 100 may be disposable. Operation of the instrument 100 begins by grasping 802 the handle 102 having distal 104 and proximal 106 ends, wherein the proximal end 106 branches to form two arm-like portions 108 and the distal end 104 widens to form an approximately tubular body portion 110. The tubular body portion 110 has surfaces 112 sized for receiving a middle finger and a thumb, wherein the branch between the two arm-like portions 108 is sized for receiving an index finger.

The user places an index finger between the two arm-like portions and then rests the pad of the index finger along the top of the device on one of the recessed portions 112, as shown in FIG. 1B. The thumb and middle finger then grasp the tubular body portion 110 of the handle 102 and the tips of the pads of the thumb and middle finger rest along the sides of the tubular body portion 110 within recessed portions 112. In this grip position, a user is utilizing the classic grip to hold the instrument 100 securely between the thumb and middle finger. The index finger along the top of the tubular body portion 110 provides more fine tuned control of the device. Utilizing this classic grip, a user can manipulate the instrument 100 using the hand, wrist and elbow and may be able to rest his/her shoulder.

Once the handle 102 is grasped 802 by a user, the method 800 of operation continues by activating 804 an actuator mechanism 120 positioned on the handle 102 to turn the electrosurgical instrument on/off. The actuator mechanism 120 may comprise a push-button, slide, or wheels and be activated using a middle finger, thumb, or index finger. In some implementations., the actuator mechanism 120 may be biased in the ‘off’ position, so that activation by a user turns the electrosurgical instrument ‘on’ (i.e., energizes the electrode 116 to create an ionized plasma gas stream for coagulation).

The method 800 of operation may further include activating a sheath actuator 140 to change an operating mode of the instrument 100. The sheath actuator 140 is operable to extend or retract the insulating sheath 118 surrounding the electrode 116. Extending or retracting the insulating sheath 118 increases or decreases the amount of electrode 116 exposed (i.e., not covered by insulating sheath 118) to change an operational mode of the instrument 100. In one implementation, the sheath actuator 140 may be activated by sliding the insulating sheath 118 manually. In other implementations, the sheath actuator 140 may be coupled to a thumb or finger slide, button, or wheel for activation by sliding, pushing, or rolling, respectively.

A person of ordinary skill in the art will appreciate further features and advantages of the devices and methods disclosed herein based on the above-described embodiments. For example, specific features from any of the embodiments described above as well as in U.S. Pat. No. 6,255,593, entitled Medical Apparatus for Generating and Ionized Gas Plasma Stream, may be incorporated into devices, systems, and/or methods disclosed herein in a variety of combinations and subcombinations, as well as features referred to in the claims below which may be implemented by means described herein. It is also anticipated that the devices and methods disclosed herein will have utility outside the field of electrosurgery.

Accordingly, the devices and methods disclosed herein are not to be limited by what has been particularly shown and described, except as indicated by the appended claims or those ultimately provided. Any publications and references cited herein are expressly incorporated herein by reference in their entirety.

What is claimed is:

1. An electrosurgical instrument, comprising:
an electrically insulated handle having distal and proximal ends, wherein the proximal end branches to form two arm-like portions and the distal end widens to form an approximately tubular body portion having features sized for receiving at least an index finger and a thumb;
gas and electrical conduits disposed within the handle for connection to external gas and electrical sources;
a hollow elongated electrode extending from the distal end of the handle and defining a gas conduit along its length, the elongated electrode operably coupled to the gas and electrical conduits;
an elongated insulating sheath enclosing the elongated electrode; and
an actuator mechanism operably coupled to the handle, the elongated electrode, and to the gas and electrical conduits, the actuator mechanism configured to alternate between at least two states; wherein a first state prevents electric current and gas from reaching the elongated electrode, and wherein a second state allows electric current and gas to reach and flow through the elongated electrode to generate and ionized plasma gas stream for electrosurgery.

2. The electrosurgical instrument of claim 1, wherein the actuator mechanism comprises a push-button switch; wherein pressing the push-button switch activates the second state and wherein releasing the push-button switch activates the first state.

3. The electrosurgical instrument of claim 1, wherein the actuator mechanism comprises a switch slidable between at least two positions; wherein the switch in a first position activates the first state and wherein the switch in a second position activates the second state.

4. The electrosurgical instrument of claim 1, wherein the actuator mechanism comprises a foot-pedal operable between at least two positions; wherein the switch in a first position activates the first state and wherein the switch in a second position activates the second state.

5. The electrosurgical instrument of claim 1, wherein the two arm-like portions are coupled at proximal ends to form a loop sized for receiving an index finger.

6. The electrosurgical instrument of claim 1, wherein the elongated electrode comprises a sharpened distal end configured for cutting when the elongated insulating sheath is retracted to expose the sharpened distal end of the elongated electrode.

7. The electrosurgical instrument of claim 1, further comprising a strain relief coupled to the tubular body portion, the strain relief coupling the gas and electrical conduits to external gas and electrical sources.

8. The electrosurgical instrument of claim 1, wherein the gas conduit is configured for operation with helium gas.

9. The electrosurgical instrument of claim 1, further comprising a sheath actuator disposed within and extending from the distal end of the handle, the sheath actuator surrounding and operably coupled to the elongated insulating sheath, wherein the sheath actuator is configured to slidably retract and extend the elongated insulating sheath to increase or decrease exposure of the elongated electrode.

10. The electrosurgical instrument of claim 9, wherein the sheath actuator is a collar sized to be slidably received within the distal end of the handle.

11. The electrosurgical instrument of claim 10, wherein the sheath actuator further comprises a switch operably coupled to the collar, the switch having at least two positions and operable to slidably retract and extend the elongated insulating sheath to increase or decrease exposure of the elongated electrode.

12. The electrosurgical instrument of claim 11, wherein the switch comprises a thumb slide.

13. The electrosurgical instrument of claim 9, wherein the sheath actuator further comprises a rotatable mechanism operable using a rotary motion to retract and extend the elongated insulating sheath to increase or decrease exposure of the elongated electrode.

14. The electrosurgical instrument of claim 9, wherein the sheath actuator further comprises a wheel mechanism operable coupled to the collar, the wheel mechanism having at least two positions and operable by spinning to retract and extend the elongated insulating sheath to increase or decrease exposure of the elongated electrode.

15. The electrosurgical instrument of claim 14, wherein the wheel mechanism is operable to adjustably retract and extend the elongated insulating sheath to adjustably increase or decrease exposure of the elongated electrode.

16. The electrosurgical instrument of claim 9, further comprising a plurality of sheath actuators operable to change an operational mode of the instrument.

17. A method of operating an electrosurgical instrument, comprising:
grasping a handle having distal and proximal ends, wherein the proximal end branches to form two arm-like portions and the distal end widens to form an approximately tubular body portion, the tubular body portion having surfaces sized for receiving a middle finger and a thumb, wherein the branch between the two arm-like portions is sized for receiving an index finger; and
activating an actuator mechanism positioned on the handle using at least one of the middle finger, thumb, or index finger to operate the electrosurgical instrument.

18. The method of claim 17, further comprising activating a sheath actuator to change an operating mode of the electrosurgical instrument.

19. An electrosurgical instrument, comprising:
an electrically insulated handle having distal and proximal ends, wherein the proximal end branches to form two arm-like portions and the distal end widens to form an approximately tubular body portion having features sized for receiving at least an index finger and a thumb;
an electrical conduit disposed within the handle for connection to an external electrical source; and
an electrode surrounded by an insulating sheath, the electrode and insulating sheath extending from the distal end of the handle and defining an electrical conduit, the electrode operably coupled to the electrical conduit and configured for electrosurgery.
